(19) United States
(12) Patent Application Publication
(10) Pub. No.: US 2002/0048823 A1
(43) Pub. Date: Apr. 25, 2002

(54) Methods and universal monoclonal antibody array

(76) Inventor: Qianjin Hu, Castro Valley, CA (US)

Correspondence Address: BEYER WEAVER & THOMAS LLP, P.O. BOX 778, BERKELEY, CA, 94704-0778, US

(21) Appl. No.: 09/929,874

(22) Filed: Aug. 13, 2001

Related U.S. Application Data
(60) Provisional application No. 60/224,854, filed on Aug. 11, 2000.

(51) Int. Cl. G01N 33/543

(52) U.S. Cl. 436/518

(57) ABSTRACT

The invention provides compositions, methods and systems that comprise or use an array of a library of monoclonal antibodies for identifying a monoclonal antibody specific for a target antigen; or for profiling a plurality of unknown antigens from a particular source such as cells, cell lysates, tissues, or animals by the antigens' monoclonal antibody binding characteristics. The invention is useful to find a monoclonal antibody for an antigen, or to characterize antigen from a particular source (e.g. a source comprising an animal having a disease) in order to develop tools for understanding a condition (e.g. the disease) in that source.

### CROSS-REFERENCES TO RELATED APPLICATIONS

This application also claims the benefit under 37 CFR 1.78 of provisional application No. 60/224,854 filed on Aug. 11, 2000. The full disclosure of the prior application is incorporated herein by reference.

### FIELD OF THE INVENTION

This invention relates to an array of monoclonal antibodies and fragments for the purpose of identifying a specific antibody for one or more antigen.

### BACKGROUND

Generating a monoclonal antibody for a particular antigen can be a powerful research tool for studying protein function and generating therapeutics. A monoclonal antibody can be used for blocking protein activity, detecting the presence of the antigen, locating the protein or antigen, and to affinity purify agents or antigens. Antibodies can also be effective therapeutic tools, for example antibodies to cancer or viral antigens are proving useful in fighting particular diseases. However, although widely used and demonstratively useful, generation of antibodies and especially of monoclonal antibodies is not an easy task. In general, it takes about 6 months and cost several thousand dollars to generate a few good monoclonal antibodies against a single antigen. The process involves obtaining a good antigen, (e.g. a protein or synthetic peptide antigen), immunizing several animals, typically mice, testing an immune response, fusing cells to create hybridomas, screening several hundred fused cells or hybridomas for production of useful antibody that binds the target antigen, and cloning a few good positive monoclonal antibodies. Sometimes after this long tedious, time-consuming procedure, still no good, useful antibody is produced.

Therefore, in the art of identifying monoclonal antibodies for particular target antigen, there is a need for a method that can increase the efficiency, speed and success of monoclonal antibody identification for a particular target antigen. In addition, it may be valuable to have a system of screening a particular antigen source (e.g. tissue or cells, or cell lysates, etc) for an antibody profile of the antigens present in the source or multiple sources. The present invention provides both these advantages and others.

### SUMMARY OF THE INVENTION

An object of the invention is to provide a method for identifying a monoclonal antibody for a given orphan antige or other target antigen. Another object of the invention is to provide a library array of monoclonal antibodies or binding fragments of monoclonal antibodies derived from one or more animals or organisms or sources for profiling a plurality of unknown antigens.

In accordance with these and other objects are provided then, a composition comprising: a solid support comprising an array of a plurality of monoclonal antibodies or binding fragments of monoclonal antibodies derived from a animal or organism having unknown specificity for one or more orphan antigens affixed to the solid support at a non-binding region of the antibody or fragment, leaving a binding region available to bind one or more orphan antigen upon contact; wherein the plurality or antibodies comprises multiple binding specificities for a plurality of orphan antigen.

Accordingly, also is provided a method of identifying a monoclonal antibody specific for an orphan antigen for which an antibody has not been identified, comprising: contacting a solid support comprising from 100 to 500,000 monoclonal antibodies or binding fragments of monoclonal antibodies affixed to the solid support with the target antigen, detecting bound target antigen on the solid support, and identifying the antibody or portion to which target orphan antigen binds.

In addition, a method of profiling a plurality of target orphan antigens derived from a particular source is provided, comprising: contacting a solid support comprising from 100 to 500,000 monoclonal antibodies or binding fragments of monoclonal antibodies having unknown specificity for antigen affixed to the solid support with a plurality of target antigens each having a tag or label for detecting the antigen bound to an antibody or fragment, detecting any bound target antigen on the solid support, and identifying the monoclonal antibody or fragment to which any tagged or labeled target antigen binds.

The invention also provides a system or kit for identifying a monoclonal antibody for a target antigen, wherein the target antigen has no known monoclonal antibody, comprising: a composition as described and instructions for screening a target orphan antigen on a solid support comprising antibodies or antibody fragments. Another system is also provided for profiling a source comprising a plurality of orphan antigen by monoclonal antibody binding characteristics, comprising: a composition as in described having an array of a library of monoclonal antibodies, and instructions and reagents for conducting an assay on a solid support for profiling the antigen source by monoclonal antibody binding using the array.

The invention also provides a composition comprising a solid support comprising an array of a plurality of monoclonal antibodies or binding fragments of monoclonal antibodies derived from one or more animals or organisms having unknown specificity for one or more antigens affixed to the solid support at a non-binding region of the antibody or fragment, leaving a binding region available to bind one or more antigen upon contact; wherein the plurality of antibodies comprises binding specificities for a plurality of antigen. Such an array can comprise a plurality of monoclonal antibodies or binding fragments of monoclonal antibodies that comprise a library of antibodies made from immunizing one or more animals with randomized peptides or natural antigens.

The invention also provides a method of comparing profiles of a plurality of antigens derived from comparable sources comprising contacting a first solid support comprising a library from 100 to 500,000 monoclonal antibodies or binding fragments of monoclonal antibodies having unknown specificity for antigen affixed to the solid support with a plurality of target antigens derived from a first source, each antigen having a tag or label for detecting the antigen bound to a monoclonal antibody or fragment, contacting a second solid support comprising the same library of monoclonal antibodies with a plurality of target antigens derived from a second comparable source, each antigen having a tag or label for detecting the antigen bound to the monoclonal antibody or fragment, detecting any bound target antigen on each solid support, and comparing a profile of binding of monoclonal antibody or fragment to any tagged or labeled target antigen from the first source and the second source. The source of antigen can be selected from the group consisting of a cell, cell lysate, tissue, body fluid, and an animal.

The first source can comprise antigen from a normal condition and the second source can comprise antigen from a diseased source.

### DETAILED DESCRIPTION OF THE PREFERRED EMBODIMENTS

The following preferred embodiments and examples are offered by way of illustration and not by way of limitation.

The composition comprises a solid support comprising an array of monoclonal antibodies or binding portions or fragments of monoclonal antibodies affixed to it. The monoclonal antibodies have generally unknown specificity for target antigen. The monoclonal antibodies or binding portions or fragments provide an array or library of monoclonal antibody against which a target antigen might find a match. The target antigen may be an orphan antigen, but also may be an antigen with a known antibody for which another, more suitable antibody is sought. The monoclonal antibodies or fragments are derived from an animal or organism. The monoclonal antibodies or fragments are affixed on the solid support in a way that retains an opportunity for the antibody to bind antigen upon contact on the solid support.

The array can comprise monoclonal antibodies or binding fragments in an amount or density in a range of about 100-100,000 antibodies or fragments per CM² of solid support. A solid support may have up to about 500,000 antibodies or fragments. However, the density or capacity of the solid support for binding antibodies or fragments is not limited to the current technology for affixing the antibodies on the solid support and for the limitations of providing binding opportunity with antigen on the space of the solid support, but rather the capacity for antibody can be increased as the technology improves. The solid support comprising monoclonal antibodies in an array can be created generally as is known in the art. For example, see WO 00/04382 and WO 00/54046.

The solid support can be glass, metal, plastic, polymer, membrane, nylon, nitrocellulose and paper. A solid substrate suitable for use in the method of the invention includes a substrate made of all or any materials on which an antibody can be immobilized for making a matrix of different antibodies for screening as described below. Thus, natural or synthetic or chemically modified or unmodified materials can be used as the solid substrate, for example polysaccharides such as cellulose based materials for example, paper, cellulose derivatives acetate and nitrocellulose, dextran, polymers such as polyvinyl chlorides, polyethylenes, polystyrenes, unsaturated carboxylic acid esters, vinylidene chloride, dienes, or compounds with nitrile groups such as acetonitrile, vinyl chloride/propylene, or vinyl chloride/acetate copolymers, natural fibers such as cotton and synthetic fibers such as nylon, inorganic materials such as silica, glass quartz, or ceramics, latexes, such as colloidal aqueous dispersions of any water-insoluble polymer, magnetic particles, metal derivatives, and other materials capable of acting as a solid substrate for this invention. The solid substrate can be in the form of a microtitration plate, a sheet, a cone, a tube, pellets, particles of some such similar configuration of the materials selected for use. The solid support can take the shape of a rectangular wafer, or some other such easily manipulatable shape which can be adapted to use by a robot in highly mechanized screening procedures. The solid support can be a microwell, for example. The shape of the solid support should lend itself to fixing the antibodies or portions or fragments onto it, and also needs to provide a grid or location system for identifying the place on the support that an antigen binds when such binding occurs. For example, a wafer or rectangular solid support may have a grid system for keeping track of which antibody is located at which coordinates, etc.

To form the composition, a plurality of monoclonal antibodies or binding portions or fragments of monoclonal antibodies derived from an animal or organism are placed (i.e. affixed or immobilized) on to the solid support. The monoclonal antibody is linked to the surface of the solid support so that it remains stable on the surface and does not shift its position on the surface. Upon hybridization with an antigen, the monoclonal antibody stays also on the solid support surface so that the monoclonal antibody or fragment binds the target antigen can be accurately identified. The antibodies are fixed onto the solid support in a manner that the binding portions of the antibodies or fragments are accessible to the antigen or to the agents that are made to contact the solid support. Thus, generally, a non-binding region of the monoclonal antibody or fragment is used to affix the monoclonal antibody to the solid support.

The antigens used in the assays comprising the solid support of monoclonal antibodies are orphan antigen or antigen for which a monoclonal antibody has not been identified, or antigen for which an antibody has been found, but for which another, more suitable antibody, is sought. Thus, the antigen can comprise polypeptides, peptides, polynucleotides, oligonucleotides, nucleic acids, carbohydrates, small molecules, polymers, lipids, fats, complexes of molecules, and other antigens and antigen molecules for which it would be desirable to find an antibody. The technology can be used as a diagnostic and as a tool for antigen profiling of a given source, answering such questions as, e.g., does this tissue (the antigen source) have a cancer antigen profile (e.g. binding monoclonal antibodies specific (generally) for cancer antigens)? The technology can also be used to find a different antibody for an antigen which has one or more antibodies already, but for which another antibody might be desirable to identify. For example, where a given previously identified antibody will not work well as a therapeutic or diagnostic antibody, it would be desirable to find another antibody for that target antigen that could perhaps work well as a therapeutic or diagnostic antibody. The technology can also be used to compare antigen profiles from two or more comparable sources of antigen. For example, a normal tissue source can be compared to a diseased tissue source in order to identify antigen differences, or antigen profiles, or the two or more sources.

To create monoclonal antibody arrays, monoclonal antibodies or binding fragments of monoclonal antibodies are spotted, placed or affixed onto a substrate in a two-dimensional matrix or array. Preferably the substrate is “sticky” for the antibody (e.g. coated with biotin and capable of binding avidin covalently linked to the antibodies). The sample antigens, orphan or otherwise, (which contact the antibody array) can be tagged or labeled for detecting the bound antigen on the array. The orphan antigen or other target antigen can be tagged or labeled using radioactive labels, fluorophors, etc. Techniques for constructing arrays for polynucleotides are instructive to constructing arrays for monoclonal antibodies, including detecting the bound target antigens on the array after contact with the monoclonal antibodies.

The arrays can be used to screen for a monoclonal antibody for one or more orphan antigen, or one or more antigen for which an antibody is sought. The arrays can also be used to profile one or more orphan antigens (or antigens with unknown antibody binding specificity) or antigens for which an antibody is sought, thus lending information about the antigen and its source. In profiling, the same plurality of antigens can be screened against several different monoclonal antibody arrays to determine which antigens are present in the plurality, e.g. viral antigens, cancer antigens, antigens characteristic of an autoimmune disorder, etc., and in general any antigen which is associated with a biological disease or condition.

The monoclonal antibodies used in the array can be whole monoclonal antibodies or binding fragments of monoclonal antibodies. Thus, for example, the antibodies can be a heavy chain, or a light chain or a portion thereof. Accordingly the antibodies can be a Fab fragment, an Fc fragment, a lambda chain, a kappa chain, or binding portions or fragments thereof. The monoclonal antibodies or fragments can be modified from a predecessor antibody, such as, for example, humanized antibodies. The monoclonal antibodies can be single chain antibodies. For example, the monoclonal antibody library for the solid support can be made from a phage display library, or a yeast display library, generated by cloning the variable regions of antibody heavy and light chains from human or animals into a phage display system to create a single chain antibody library. The monoclonal antibodies are modified to permit binding to the solid support.

The solid support comprises enough monoclonal antibodies (e.g. in the range from about 100 to about 500,000 antibodies per cm²) to provide a diversity of binding molecules for the target antigens being screened. Multiple solid supports comprising in excess of 500,000 monoclonal antibodies in total can be used to screen for a monoclonal antibody specific for the antigen targets. Each monoclonal antibody will possess a unique binding specificity in order to provide the largest diversity of binding opportunities possible in the screening process. Any antigen for which it is desired to establish or find a monoclonal antibody can be screened on the solid support. The antigen may be an orphan antigen, or an antigen for which no known binding antibody exists, or the antigen may be any target antigen for which it would be desirable to find other antibody binding molecules. For example, a toxin or viral antigen or other harmful or disease causing agent for which no known mononclonal antibody exists, and for which it would be desirable to discover a monoclonal antibody.

Immunogens for raising monoclonal antibodies can be prepared by mixing polypeptides or peptides or other natural antigens with adjuvants. Alternatively, polypeptides or peptides or other antigens can be made as fusion proteins to larger immunogenic proteins. Polypeptides can also be covalently linked to other larger immunogenic proteins, such as keyhole limpet hemocyanin. Immunogens are typically administered intradermally, subcutaneously, or intramuscularly. Immunogens are administered to experimental animals such as rabbits, sheep, and mice to generate antibodies. Optionally, the animal spleen cells can be isolated and fused with myeloma cells to form hybridomas which secrete monoclonal antibodies. Such methods are well known in the art. According to another method known in the art, a polynucleotide encoding the antigen is administered directly such as by intramuscular injection and expressed in vivo. The expressed protein generates a variety of protein-specific immune responses, including production of antibodies comparable to the administration of the protein. Preparations of polyclonal and monoclonal antibodies for protein antigens, polypeptides, peptides, polysaccharide, and other antigens are made using standard methods known in the art. The antibodies specifically bind to epitopes present in the polypeptides or peptide antigen. Typically, at least 6, 8, 10, or 12 contiguous amino acids are require to form an epitope. However, epitopes which involve non-contiguous amino acids may require more, for example, at least 15, 25, or 50 amino acids.

The plurality of monoclonal antibodies on the solid support can be monoclonal antibodies or binding fragments made from immunizing a mammal (e.g. a rodent, a dog, a cat, a pig, a horse, a cow, etc.) with peptides (e.g. randomized peptides), natural antigens, or any antigen for which it is desired to find a monoclonal antibody. A library of peptides may be synthesized to generate antibodies following the methods disclosed in U.S. Pat. No. 5,010,175 and in PCT WO91/17823.

The monoclonal antibodies can be generated from a source for example selected from the group consisting of yeast, insect, worm, fish, avian, worm, mammal, and rodent, or in general any animal or organism source available to generate monoclonal antibodies. Multiple animals may be represented on an array of monoclonal antibodies, or a single animal or organism may be represented on an array of monoclonal antibodies.

The monoclonal antibody generating animal is injected with the orphan antigens and antibodies are produced within the animal and harvested sometime later after the antibodies have had a chance to develop and mature. Each solid support can be classified by the antibody source. For example, the same immunogen (antigens) can be injected into several different species of animals, and a solid support matrix can be generated from each species.

Monoclonal antibodies for the solid support can be individually produced, as described herein, and by methods well known in the art. Antibodies for the solid support can be individually purified by methods well known in the art. For example, the monoclonal antibodies are affinity purified by passing antiserum over a column to which the antigen that generated the antibody is bound. The bound antibodies can be eluted from the column, for example using a buffer with a high salt concentration. A single solid support can have monoclonal antibodies from a single organism or animal, or the solid support can have monoclonal antibodies from multiple organism or animal sources.

Monoclonal antibodies that specifically bind to a target antigen or orphan antigen on the solid support should provide a detection signal at least 5-, 10-, or 20-fold higher than a detection signal provided with any background or non-specific binding on the solid support. Ultimately, an antibody that is detected as specific for a target antigen will not detect other proteins in an immunochemical assay and can immunoprecipitate the antigen in a subsequent assay.

The invention provides methods of identifying a monoclonal antibody specific for an antigen for which an antibody has not been identified. A solid support of a plurality of different monoclonal antibodies having different but unknown binding specificities (formed and presented in an array on a solid support) is contacted with the target (orphan or known) antigen for which it is desirable to find an antibody binding partner. The bound target antigen is detected on the solid support, e.g. by detecting a tag or label placed on the antigen before it contacts the antibody array. The array of antibodies is constructed as described above (e.g. having in a range from about 100 to about 500,000 antibodies on a given array or matrix. The bound target or orphan antigen is detected by virtue of detecting the tagged or labeled antigen on the array at a particular coordinate. This detection identifies the monoclonal antibody that binds the antigen. The antibody can then be further characterized and produced (e.g. recombinantly) and re-tested for binding to the orphan antigen. The discovered monoclonal antibody can be further refined, determining if desirable the binding region of the antibody and/or antigen. The monoclonal antibody can be refined also, for therapeutic or diagnostic use, e.g. modified to a single chain or humanized antibody for human use.

The invention provides also a method of profiling an antigen source by the monoclonal antibody binding characteristics or pattern of the antigens derived from that source. The pattern of antigens is determined by which antigens bind a particular monoclonal antibody array, or conversely by which antibodies bind the antigens derived from that source. A monoclonal antibody array as described above is made and the array or solid support matrix is contacted with a plurality of target antigen from a source. The target antigen is tagged with a tag or labeled with a label as is known in the art for preparing targets for detection on solid support arrays. All the antigens may have the same tag or label due the ability to reference bound entities on the array. Alternatively, the antigens can have different tags or labels. After the monoclonal antibody that binds antigen is identified by its location on the matrix array, the bound target antigen can then be characterized by the antibody it binds, and the source further characterized by this binding pattern. For example, a particular disease or biological condition may have a monoclonal antibody binding pattern. The source of the antigen can be, for example, a cell, a cell lysate, a tissue, body fluid, or an animal. The animal can be a mammal. In general, the source can be any source comprising one or more antigen for which it is desirable to elucidate a pattern of monoclonal antibody binding.

A composition comprising a solid support comprising an array of a plurality of monoclonal antibodies or binding fragments of monoclonal antibodies derived from one or more animals or organisms having unknown specificity for one or more antigens is provided. The antibodies are affixed to the solid support at a non-binding region of the antibody or fragment, leaving a binding region available to bind one or more antigen upon contact. The plurality of antibodies can comprise binding specificities for a plurality of antigen. Such an array can comprise a plurality of monoclonal antibodies or binding fragments of monoclonal antibodies that comprise a library of antibodies made from immunizing one or more animals with randomized peptides or natural antigens. Multiple sources of antibodies can be used on a single array. For example, different animals, organisms, tissues, or other sources of antibodies can be combined to form a particular array.

In addition, a method of comparing profiles of a plurality of antigens derived from comparable sources is provided. The method comprises contacting a first solid support comprising a library from 100 to 500,000 monoclonal antibodies or binding fragments of monoclonal antibodies having unknown specificity for antigen affixed to the solid support with a plurality of target antigens derived from a first source, each antigen having a tag or label for detecting the antigen bound to a monoclonal antibody or fragment. In a parallel array, a second solid support (with the same antibodies as the first solid support) is provided. The second solid support comprises the same library of monoclonal antibodies. The second solid support is contacted with a plurality of target antigens derived from a second source which is a source that is comparable to the first antigen source. Each antigen has a tag or label for detecting the antigen bound to the monoclonal antibody or fragment. After contact, the method comprises detecting any bound target antigen on each solid support, and comparing a profile of binding of monoclonal antibody or fragment to any tagged or labeled target antigen from the first source and the second source. The sources of antigen can be selected from the group consisting of a cell, cell lysate, tissue, body fluid, an organism and an animal. For example, the first source can comprise antigen from a normal condition and the second source can comprise antigen from a diseased source.

Systems or kits are also provided by the invention employing the compositions (monoclonal antibody arrays) described above, and instructions for either screening the arrays for locating parent monoclonal antibodies for orphan antigens, or for screening a plurality of antigen from a source for characteristic monoclonal antibody binding profiles, as described above.

The invention is practiced by generating an array comprising a library of monoclonal antibodies affixed to a solid support. The position of each monoclonal antibody is recorded so that the grid embodies necessary information for locating an antibody that binds to an antigen. The array is contacted with one or more orphan or target antigens and the position of any antigen that binds an antibody on the array is retraced to the location on the grid. The identified monoclonal antibody can then be further tested for binding affinity to the antigen. A plurality of antigen can be profiled, e.g. where the antibodies on the array are selected for the information they can impart about the antigen content being tested. For example, an array of monoclonal antibody can comprise, e.g. monoclonal antibodies to cancer antigens, or monoclonal antibodies to antigens generated in any disease condition.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A composition comprising:
a solid support comprising an array of a plurality of monoclonal antibodies or binding fragments of monoclonal antibodies derived from an animal or organism having unknown specificity for one or more orphan antigens affixed to the solid support at a non-binding region of the antibody or fragment, leaving a binding region available to bind one or more orphan antigen upon contact; wherein the plurality of antibodies comprises binding specificities for a plurality of orphan antigen.

2. A composition as in claim 1, wherein the array comprising the plurality of monoclonal antibodies or binding fragments of monoclonal antibodies is in a range from about 100 to about 100,000 antibodies or portions per CM² of the solid support.

3. A composition as in claim 1, wherein the solid support comprises a material selected from the group consisting of glass, metal, plastic, polymer, membrane, nylon, nitrocellulose and paper.

4. A composition as in claim 1, wherein the array comprising the plurality of monoclonal antibodies or binding fragments of monoclonal antibodies comprise a library of antibodies made from immunizing a mammal with randomized peptides or natural antigens.

5. A composition as in claim 1, wherein the array comprising the plurality of monoclonal antibodies or binding fragments of monoclonal antibodies comprises a library of monoclonal antibodies made from a phage display library generated by cloning the variable regions of antibody heavy and light chains from human or other animals into a phage display system to create a single chain monoclonal antibody library on the array.

6. A composition as in claim 1, wherein the array comprising the plurality of monoclonal antibodies or binding fragments of monoclonal antibodies comprises one or more single chain antibodies.

7. A composition as in claim 1, wherein the array comprising the plurality of monoclonal antibodies or binding fragments of monoclonal antibodies comprises monoclonal antibodies generated from a source selected from the group consisting of yeast, insect, worm, fish, avian, mammal, rodent, cells, cell lysates and tissues.

8. A composition as in claim 1, wherein the monoclonal antibodies or binding fragments of monoclonal antibodies are individually produced.

9. A composition as in claim 1, wherein the monoclonal antibodies or binding fragments of monoclonal antibodies are individually purified.

10. A method of identifying a monoclonal antibody specific for an orphan antigen for which an antibody has not been identified, comprising:
contacting from 100 to 500,000 monoclonal antibodies or binding fragments of monoclonal antibodies affixed to a solid support with the target orphan antigen,
detecting bound target orphan antigen on the solid support, and
identifying the monoclonal antibody or portion to which the target orphan antigen binds.

11. A method as in claim 10, wherein the target orphan antigen comprises a tag or label for detecting the antigen bound to a monoclonal antibody.

12. A method of profiling a plurality of unknown antigens derived from a particular source comprising:
contacting from 100 to 500,000 monoclonal antibodies or binding fragments of monoclonal antibodies having unknown specificity for antigen affixed to the solid support with a plurality of target antigens each having a tag or label for detecting the antigen bound to a monoclonal antibody or fragment,
detecting any bound target antigen on the solid support, and
identifying the monoclonal antibody or fragment to which any tagged or labeled target antigen binds.

13. A method as in claim 10 or 12, wherein the source is selected from the group consisting of a cell, cell lysate, tissue, body fluid, and an animal.

14. A kit for identifying a monoclonal antibody for a target orphan antigen, wherein the target antigen has no known monoclonal antibody, comprising:
a composition as in claim 1, and instructions for screening a target orphan antigen on a solid support comprising monoclonal antibodies or fragments.

15. A kit for profiling a source comprising a plurality of orphan antigen by monoclonal antibody binding characteristics, comprising:
a composition as in claim 1, reagents for conducting an assay on a solid support for profiling the antigen source by monoclonal antibody binding, and instructions for conducting the assay.

16. A composition comprising:
a solid support comprising an array of a plurality of monoclonal antibodies or binding fragments of monoclonal antibodies derived from one or more animals or organisms having unknown specificity for one or more antigens affixed to the solid support at a non-binding region of the antibody or fragment, leaving a binding region available to bind one or more antigen upon contact; wherein the plurality of antibodies comprises binding specificities for a plurality of antigen.

17. A composition as in claim 16, wherein the array comprising the plurality of monoclonal antibodies or binding fragments of monoclonal antibodies comprise a library of antibodies made from immunizing one or more animals with randomized peptides or natural antigens.

18. A method of comparing profiles of a plurality of antigens derived from comparable sources comprising:
contacting a first solid support comprising a library from 100 to 500,000 monoclonal antibodies or binding fragments of monoclonal antibodies having unknown specificity for antigen affixed to the solid support with a plurality of target antigens derived from a first source, each antigen having a tag or label for detecting the antigen bound to a monoclonal antibody or fragment,
contacting a second solid support comprising the same library of monoclonal antibodies with a plurality of target antigens derived from a second comparable source, each antigen having a tag or label for detecting the antigen bound to the monoclonal antibody or fragment,
detecting any bound target antigen on each solid support, and
comparing a profile of binding of monoclonal antibody or fragment to any tagged or labeled target antigen from the first source and the second source.

19. A method as in claim 18, wherein the source is selected from the group consisting of a cell, cell lysate, tissue, body fluid, an organism and an animal.

20. A method as in claim 18, wherein the first source comprises antigen from a normal condition and the second source comprises antigen from a diseased source.
